(19) United States
(12) Patent Application Publication
(10) Pub. No.: US 2025/0164404 A1
(43) Pub. Date: May 22, 2025

(54) SYSTEM AND METHOD FOR ENHANCED ROCK WEATHERING MONITORING, REPORTING, AND VERIFICATION

(71) Applicants: Impossible Sensing LLC, St. Louis, MO (US); Mati Carbon PBC, Wilmington, DE (US)

(72) Inventors: Pablo Sobron, St. Louis, MO (US); Jacob S. Jordan, St. Louis, MO (US); Shantanu Agarwal, Houston, TX (US)

(21) Appl. No.: 18/954,208

(22) Filed: Nov. 20, 2024

Related U.S. Application Data
(60) Provisional application No. 63/601,059, filed on Nov. 20, 2023.

(51) Int. Cl. G01N 21/71; G01N 33/24

(52) U.S. Cl. CPC G01N 21/718; G01N 33/241; G01N 33/245; G01N 2201/0216; G01N 2201/0221; G01N 2201/06113; G01N 2201/12746

(57) ABSTRACT

A method and system for monitoring, reporting, and verification of enhanced rock weathering is disclosed. In some embodiments, the method includes emitting, using a spectroscopy device, one or more optical signals directed to a target collected from a target area. The method includes determining, using the spectroscopy device, spectral measurements of the optical signals collected from the target. The method includes converting, using a data analysis subsystem, the spectral measurements to data indicating compositional characteristics of the target. The method also includes analyzing, by the data analysis subsystem, the data indicating compositional characteristics of the target using a plurality of analytical tools. The method further includes providing, using the data analysis subsystem, assessments on carbon dioxide removal and insights on enhanced rock weathering.

### CROSS REFERENCE TO RELATED APPLICATION

This application claims the benefit of U.S. Provisional Patent Application No. 63/601,059, titled “System and Method for Enhanced Rock Weathering Monitoring, Reporting, and Verification,” and filed on Nov. 20, 2023, the entire content of which is incorporated by reference herein.

### TECHNICAL FIELD

This disclosure relates to compositional characterization techniques, in particular, to optical devices that embody new methodologies for monitoring, reporting, and verification (MRV) of enhanced rock weathering (ERW).

### BACKGROUND

Enhanced rock weathering (ERW) is the practice of applying crushed rocks and silicate minerals to agricultural land. It is a promising scalable and cost-effective carbon dioxide removal (CDR) strategy with significant environmental and agronomic co-benefits. However, measuring the extent and progress of carbon capture is difficult due to the complexity of soil properties and other conditions. Most current projects/systems/devices cannot accurately obtain carbon measurement and properly report carbon sequestration. Also, ERW lacks a robust monitoring, reporting, and verification (MRV) framework to address challenging problems such as carbon deception. For example, current MRV practices cannot provide a framework to easily detect and process a fraudulent claim of carbon gains by dishonest actors in the system.

### SUMMARY

To address the aforementioned shortcomings, a method and a system for monitoring, reporting, and verification of enhanced rock weathering is disclosed. The method include using a spectroscopy device to emit one or more optical signals directed to a target collected from a target area and to determine spectral measurements of the optical signals collected from the target. The method also includes using a data analysis subsystem to convert the spectral measurements to data indicating compositional characteristics of the target, analyze the data indicating compositional characteristics of the target using a plurality of analytical tools, and provide assessments on carbon dioxide removal and insights on enhanced rock weathering.

To convert the spectral measurements to the data indicating compositional characteristics of the target, one or more of the processes, such as determining a weathering status, evaluating target contents, calculating heavy metal concentration, determining metal ratios in the target, or measuring organic carbon in the target, are performed. To determine the weathering status, loss of rock dust is determined by assessing the composition of cations in rock dust applied to the target area through the enhanced rock weathering. To analyze the data indicating compositional characteristics, analytical tools are used to perform one or more of multivariate distribution modeling for cation covariance analysis, empirical modeling for weathering prediction and carbon credit creation, calibration with high-precision inductively coupled plasma mass spectrometers (ICP-MS) data, construction of calibration curves for soil-rock dust mixtures with high-precision ICP-MS, and collaborative spot-checking and uncertainty modeling with ICP-MS and LIBS. To provide the assessment on carbon dioxide removal and insights on enhanced rock weathering, a prediction model is created to provide data support for subsequent implementation of enhanced rock weathering, and/or one or more of an enhanced weathering potential and a total carbon dioxide removal (CDR) potential are determined.

In some embodiments, the heavy metal concentration is calculated in response to receiving bio-availability of metals and dissolved aqueous metals. In some embodiments, the soil organic carbon is measured based on determining the loss of material on ignition. In some embodiments, the target is a pooled sample that includes soil samples collected and combined from multiple locations within the target area. One or more geolocated zones within the target area may be identified from which the pooled sample is collected, and the pooled sample is collected from the target area using grid-based or rastered laser induced breakdown spectroscopy (LIBS) sampling. In some embodiments, the spectroscopy device is a handheld LIBS device. In some embodiments, the spectroscopy device is a LIBS device that is mounted on a motor vehicle and operates in real time as the motor vehicle traverses the target area, and the data analysis subsystem is integrated onboard the motor vehicle for real-time data acquisition and analysis. In some embodiments, the spectroscopy device is a LIBS device that is combined with a soil auger to obtain a depth-integrated soil profile of a predetermined target characteristic from the target.

The above and other preferred features, including various novel details of implementation and combination of elements, will now be more particularly described with reference to the accompanying drawings and pointed out in the claims. It will be understood that the particular methods and apparatuses are shown by way of illustration only and not as limitations. As will be understood by those skilled in the art, the principles and features explained herein may be employed in various and numerous embodiments.

### BRIEF DESCRIPTION OF THE DRAWINGS

The disclosed embodiments have advantages and features which will be more readily apparent from the detailed description, the appended claims, and the accompanying figures (or drawings). A brief introduction of the figures is below.

FIG. 1A illustrates a block diagram of an exemplary ERW process.

FIG. 1B illustrates a graphic representation of the speciation of a dominant form of dissolved carbon.

FIG. 2A illustrates an overview of an exemplary laser induced breakdown spectroscopy (LIBS)-based data system, according to some embodiments.

FIG. 2B illustrates an overall diagram of an exemplary LIBS device, according to some embodiments.

FIG. 3 illustrates a block diagram of exemplary uses of LIBS devices in an MRV process for ERW, according to some embodiments.

FIG. 4 illustrates a block diagram of exemplary features of LIBS devices and analysis used in MRV for ERW described in the present system, according to some embodiments.

FIG. 5 illustrates a block diagram of exemplary data analysis and modeling used in the present system, according to some embodiments.

FIG. 6 illustrates a flowchart 600 of monitoring, reporting, and verification of enhanced rock weathering, according to some embodiments.

FIG. 7 illustrates a block diagram of an example computer system that may be used in implementing the technology described herein, according to some embodiments.

### DETAILED DESCRIPTION

The Figures (FIGs.) and the following description relate to preferred embodiments by way of illustration only. It should be noted that from the following discussion, alternative embodiments of the structures and methods disclosed herein will be readily recognized as viable alternatives that may be employed without departing from the principles of what is claimed.

Reference will now be made in detail to several embodiments, examples of which are illustrated in the accompanying figures. It is noted that wherever practicable similar or like reference numbers may be used in the figures and may indicate similar or like functionality. The figures depict embodiments of the disclosed system (or method) for purposes of illustration only. One skilled in the art will readily recognize from the following description that alternative embodiments of the structures and methods illustrated herein may be employed without departing from the principles described herein.

### Scientific Introduction

### ERWA—A Durable Carbon Dioxide Removal Strategy

Enhanced rock weathering (ERW) is a carbon dioxide removal (CDR) technique where naturally occurring mineral weathering reactions that consume atmospheric CO₂ are accelerated. This may be achieved by applying crushed rocks and silicate minerals to agricultural soils. When the gradual rock dissolution or “weathering” occurs, alkaline minerals containing cations, such as calcium and magnesium (Ca²⁺ and Mg²⁺) that comprise the silicates, act as a pH amendment to remediate acidic soil on farms. In addition to balancing the pH of the soil, ERW can remove carbon dioxide from the atmosphere and sequester carbon for geological timescales.

Basalt can be applied to managed land or an agricultural setting (e.g., rice paddies, corn fields, pasture lands) to practice ERW as a CDR strategy at scale. It should be recognized that the approach described herein is crop agnostic and can be generally applicable to any agricultural or managed land. As the dissolution of basalt proceeds, CO₂ gas in the soil is converted to dissolved bicarbonate (HCO₃⁻) in groundwater. This occurs due to basalt weathering reactions involving carbonic acid. Carbonic acid (H₂O+CO₂═H₂CO₃) is formed naturally in soils by plant respiration. The conversion of dissolved carbonic acid sources to bicarbonate draws CO₂ down from the atmosphere.

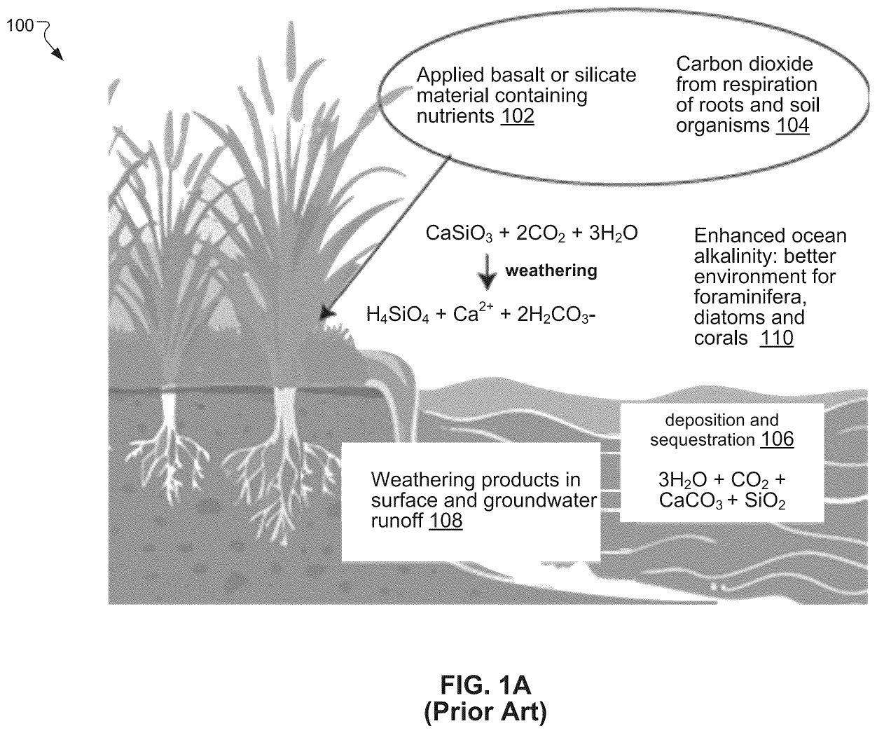

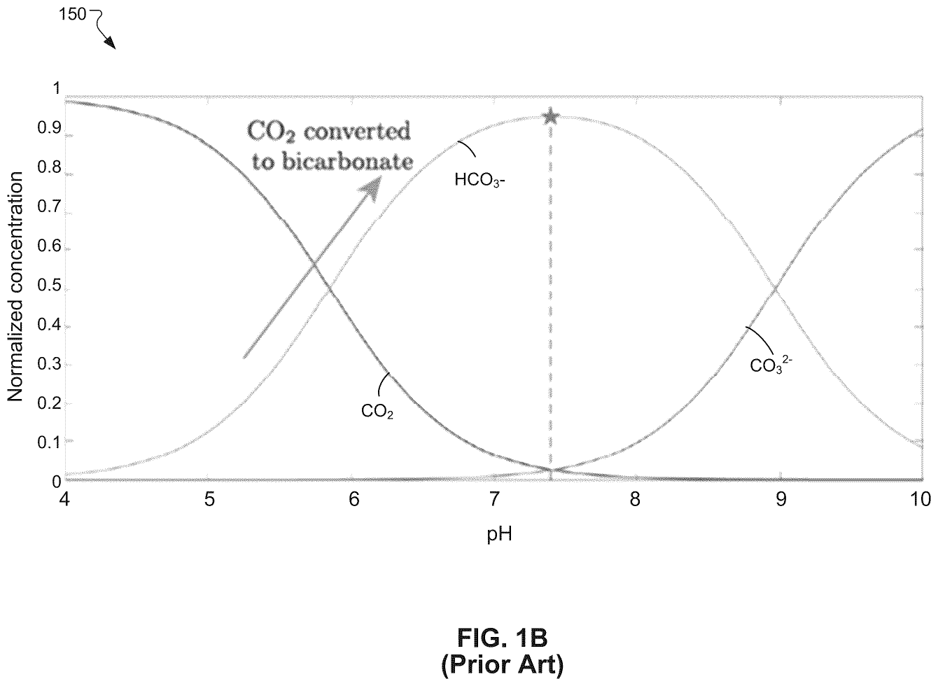

FIG. 1A illustrates a block diagram 100 of an exemplary ERW process. Basalt and/or other silicate materials (e.g., crushed rocks or “rock powder”) are applied to the soil and mixed into the subsurface near crops in 102. As the ERW progresses, the carbon dioxide (CO₂ gas) from the respiration of roots 104 is converted to bicarbonate (HCO₃⁻) in groundwater, and the CO₂ stored in the soil 104 is also leached out of the soil and converted to bicarbonate. The bicarbonate is associated with a rising pH value of soil since the soil becomes less acidic. In this way, CO₂ is removed. The silicate material application and CO₂ isolation are associated with the deposition and sequestration processes 106, which results in the formation of weathering products in surface and groundwater runoff 108. Multiple enhanced rock weathering experiments have also demonstrated significant agronomic benefits due to improved nutrient supply for crops. Therefore, ERW deployments could aid farmers in regions where fertilizers are scarcely available or unaffordable.

### Carbon in Agricultural Setting

The behavior of carbon in agricultural settings depends on the pH values in the settings. FIG. 1B illustrates a graphic representation 150 of the speciation of a dominant form of dissolved carbon, specifically, a representative calculation for the speciation of dissolved inorganic carbon (DIC). DIC is the sum of the aqueous species of inorganic carbon in a solution, which includes three major aqueous species: CO₂, HCO₃⁻, and CO₃²⁻. The speciation of DIC is the distribution of these species in a solution. The speciation of DIC can be determined by measuring the pH and total alkalinity of a solution and then using the measured values to calculate the concentrations of the different DIC species.

To obtain the calculation demonstrating how the behavior of DIC changes with the pH value of soil in FIG. 1B, the pH value of the soil and pore waters of rice paddies is shifted towards pH=7 from ERW. As low pH (i.e., acidic) soils transition to neutral pH (i.e., pH≈7 ), dissolved CO₂ is converted to the alkaline dissolved mineral bicarbonate (HCO₃⁻). Near PH=7 bicarbonate is the dominant form of DIC. In other words, based on the shifting of the pH value, CO₂ is converted to HCO₃⁻ and is not released as gas into the atmosphere.

Next it is investigated what happens after CO₂ is converted to other forms of dissolved inorganic carbon. After the conversion from CO₂ to HCO₃⁻, the bicarbonate (HCO₃⁻) percolates into streams and rivers, from where the bicarbonate is delivered to the oceans. This DIC may reside in groundwater within the soil column for decades to centuries. This is an effective removal of carbon dioxide available to the ocean-atmosphere system. The eventual precipitation of bicarbonate as calcium carbonate minerals in deep aquifers or after transport to the ocean stably stores carbon for more than 10,000 years. The delivery of DIC to the ocean in the form of bicarbonate acts to enhance ocean alkalinity, which is vital for the health of ocean flora and fauna. For example, coral ecosystems have been particularly damaged by ocean acidification which is a well-known side effect of climate change. Ocean alkalinity enhancement works to counteract ocean acidification, as shown in 110 of FIG. 1A.

### Technical Problems

It is hard to measure carbon capture. In agricultural and forest environments, many methods have been developed for measuring how much carbon is stored in soil. These methods significantly rely on labor-intensive mass-spectrometer measurements, spectroscopy, satellite-based sensors, and modeling. However, understanding the ecosystematic changes in soils is complex, as conditions may vary across a single plot of land. These methods cannot accurately evaluate carbon captured and retained in soils. Most current projects/methods cannot successfully quantify the amount of carbon, much less report carbon sequestration and conservation confidently. These schemes do not help address or incentivize emission avoidance and reduction by high-carbon industries (including agriculture).

Even if the issue of carbon measurement can be solved, there are other challenges such as carbon deception. This is another main challenge related to the “net zero” demand in ecosystems. “Net zero” refers to a situation where an amount of carbon dioxide is removed from the atmosphere after that amount of carbon dioxide emission is generated, resulting in zero increase in net emissions. Thus, gas emission is still generated from human activity (e.g., fossil fuel companies can continue to explore, drill, extract, and burn fossil fuels), but carbon dioxide is taken out of the atmosphere in certain ways in some other places to balance out emissions. However dishonest actors may present during this procedure. For example, like a manufacturer rigging vehicles to cheat on emissions tests, forestry interventions may cut trees and then replant trees to claim carbon gains, i.e., carbon deception. Monitoring, reporting, and verification (MRV) is the process that can account for, communicate, and certify a CDR system's carbon removal over time. Currently, ERW lacks a robust MRV framework to obtain proper knowability of carbon sequestration (e.g., detecting carbon deception) and fill the knowledge gap to fulfill the demand of “net zero.”

MRV is technically difficult due to the long timescales and uncertainties associated with the chemical and physical processes involved. Some crucial issues in MRV include accurately tracking carbon uptake, ensuring the permanence of carbon storage (e.g., long-time leakage risk), dealing with the variability of the process across different conditions, developing standardized reporting methods that ensure credibility and comparability across projects, etc. For example, there is no feasible way to directly measure the amount of CO₂ removed from the atmosphere by ERW, and measuring CO₂ in soils and groundwater is inherently challenging due to variability in environmental conditions (e.g., temperature, moisture levels, soil type, microbial activity). Emerging technologies (e.g., remote sensing, advanced soil sampling techniques) and research on effective MRV may help overcome some of the challenges.

### Overview

The present disclosure proposes a novel system and method for performing compositional characterization of soil-rock dust mixtures. In some embodiments, the present system includes laser induced breakdown spectroscopy (LIBS) instruments that embody new MRV methodologies for ERW. LIBS deduces the elemental composition of a sample (e.g., solids, liquids, and gases) from measurements of the spectral line emission emitted by the plasma produced by high-power laser irradiation of the sample. LIBS will be described in further detail below.

In some embodiments, the proposed workflow as described herein may include four primary facets:
i. Choose where to sample and measure compositional characteristics of soils;
ii. Determine how to measure compositional characteristics of the soils using LIBS;
iii. Analyze LIBS measurements (e.g., based on existing gold-standard measurement techniques); and
iv. Use post-processing analytical tools to create high-quality, verifiable carbon credits (a tradable certificate used to reduce global greenhouse gas emissions).

The present disclosure also proposes multiple LIBS devices that may be employed in the field, workstations, or laboratories.

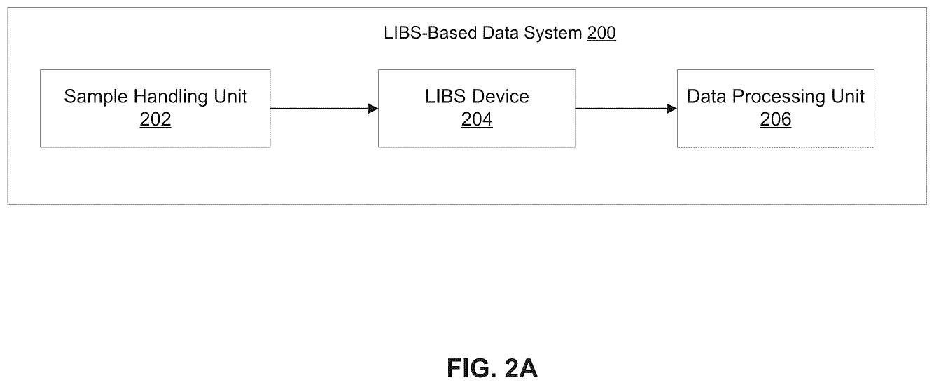

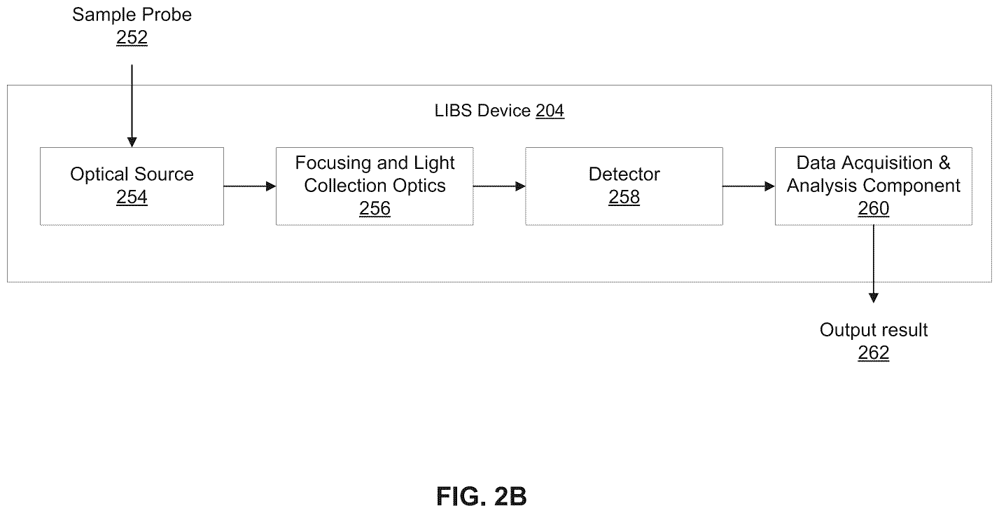

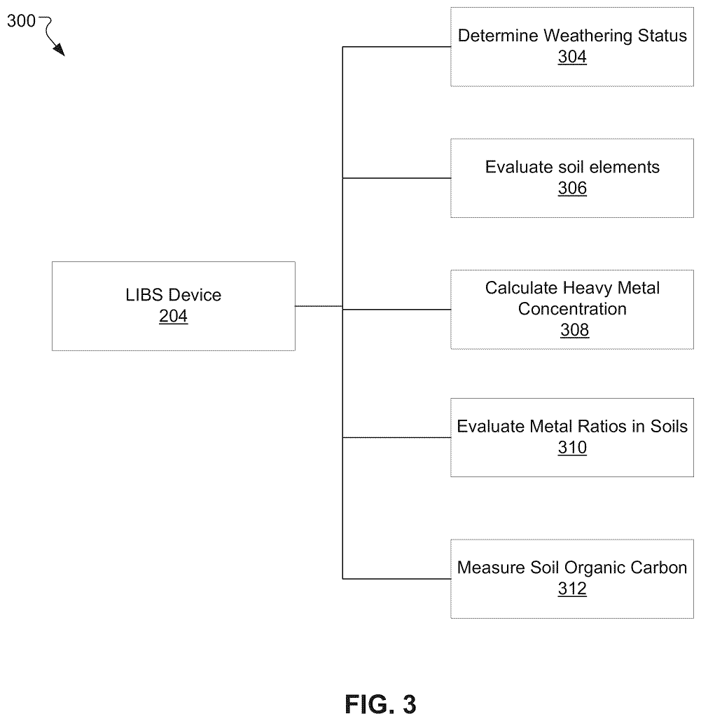

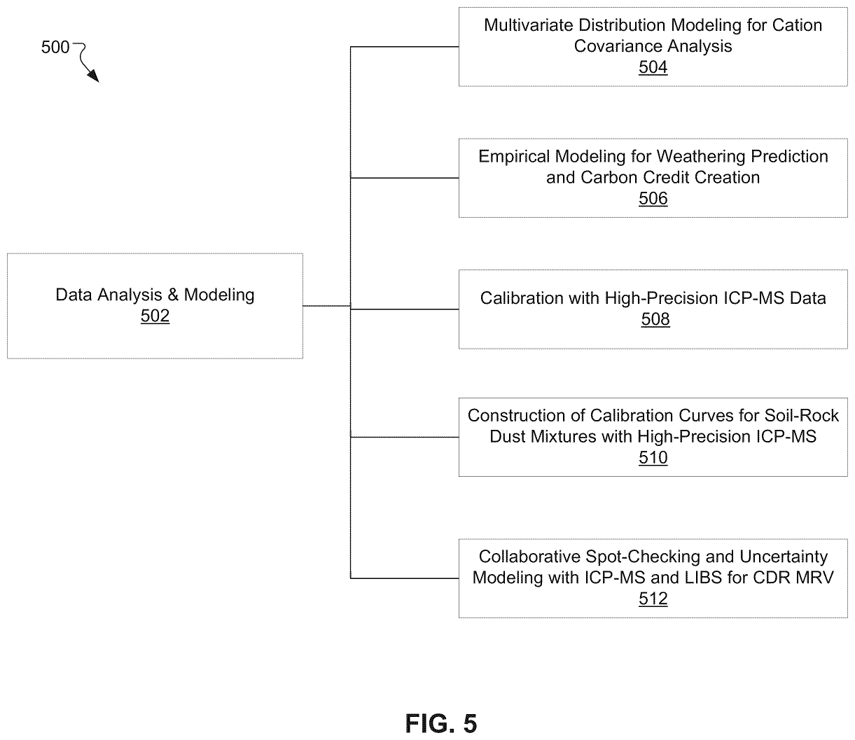

FIG. 2A illustrates an overview of an exemplary LIBS-based data system 200 that is used to implement the functionalities described herein. In this example, system 200 includes a sample handling unit 202, a LIBS device 204, and a data processing unit 206. System 200 may use sample handling unit 202 to identify locations or zones that target samples (e.g., soil samples) should be collected from (e.g., geolocated zone-based soil sampling). Depending on different quantitative analyses and qualitative analyses to be performed (e.g., as described in FIGS. 3-6), sample handling unit 202 may also be used to instruct LIBS device 204 to obtain specific types of measurements. The present system may further use sample handling unit 202 to ensure the accurate positioning of a sample for optical interaction with LIBS device 204. LIBS device 204 is employed to obtain measurements and analysis data from samples, which will be described in detail in FIG. 2B. The data from LIBS device 204 is further analyzed and modeled by data processing unit 206 for the purpose of monitoring, reporting, and verification, for example, as illustrated in FIG. 5 below. Although only one LIBS device is shown in the example system 200 of FIG. 2A, it is possible that more LIBS devices and/or other units/components may be included in a LIBS-based data system.

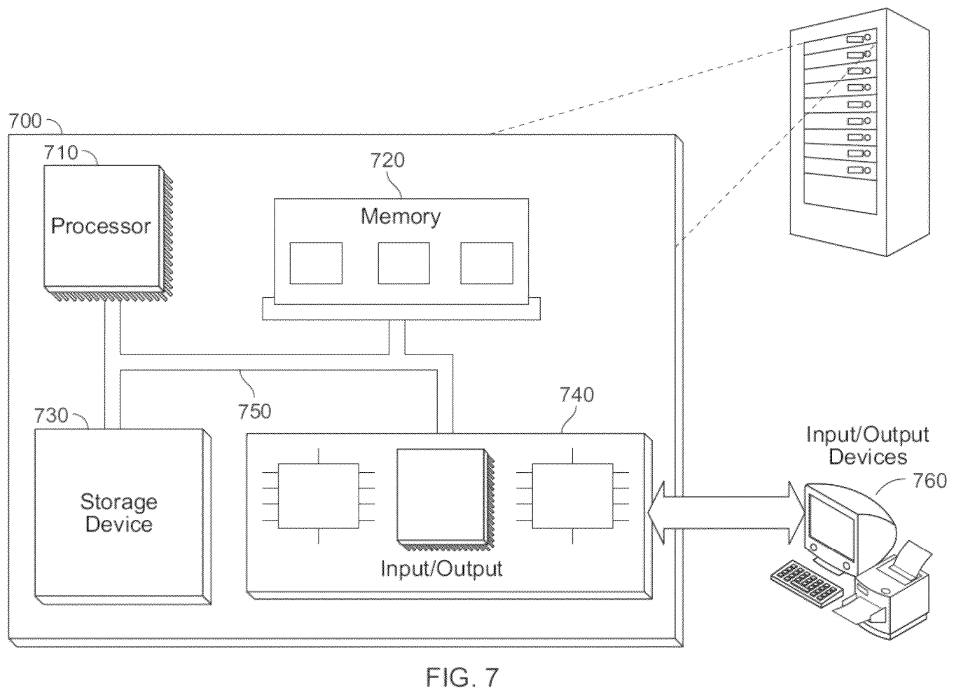

In some embodiments, at least a portion of the data processing unit 206 is part of a computer system (described below in FIG. 7) that is communicatively coupled to LIBS device 204. In other embodiments, at least a portion of the data processing unit 206 may be integrated into LIBS device 204. For example, data processing unit 206 may reside on a standalone computer system to perform advanced compositional analysis based on real-time sample analysis from data acquisition & analysis component 260 of LIBS device 204 (described below in FIG. 2B). Data processing unit 206 together with data acquisition & analysis component 260 form a data analysis subsystem that is responsible for various types of data processing, evaluation, calculation, etc, as described in the present disclosure. When LIBS device 204 is attached to or mounted on a motor instrument (e.g., vehicle, drone), the data analysis subsystem may be integrated into data acquisition onboard the motor instrument for real-time data acquisition and analysis. In some embodiments, at least a portion of the sample handling unit 202 may also be part of an external computer system and/or an integrated part of LIBS device 204. For example, sample handling unit 202 may be designed to use motorized stages or robots to move a sample in a controlled manner and thus is included in or attached to LIBS device 204 for precise sample positioning.

### LIBS Device

LIBS is a rapid chemical analysis technology that uses a short laser pulse to create a micro-plasma on the sample surface. LIBS can be used to detect the presence of various elements in a sample based on directing a high-power emission from a laser onto the sample to form a plasma. The plasma is then analyzed spectroscopically to determine the composition of the sample. The LIBS is often used with a soil penetrating probe to detect heavy metal contamination in soil due to its high sensitivity and sample-free preparation. The LIBS analytical technique offers some other compelling advantages such as fast measurement time (e.g., a few seconds for a single spot analysis), broad elemental coverage (e.g., including, but not limited to, lighter elements such as H, Be, Li, C, N, O, Na, and Mg), versatile sampling protocols that include fast raster of the sample surface and depth profiling, etc.

The present disclosure proposes new LIBS device(s) used in the MRV process for ERW. In some embodiments, the new LIBS device is a single multi-purpose optical device. In other embodiments, the new LIBS device may include one or more optical devices. Each device can be designed for a single purpose or multiple purposes. The LIBS devices may have various physical components and/or configurations.

FIG. 2B illustrates an overall block diagram of an exemplary LIBS device 204, which includes some key components such as optical source 254, focusing and light collection optics 256, detector 258, and data acquisition & analysis component 260. These components are depicted merely for illustration, and other components (more or less) may be included in LIBS device 204. For example, sample probe 252 may be part of the LIBS device 204. In some embodiments, LIBS device 204 may use a sample probe 252 (e.g., a soil penetrating probe) to take a sample from a target area that is strategically identified (e.g., on managed land). In response, optical source 254 (e.g., a laser) may generate a high-energy light pulse that is directed onto the sample, and focusing and light collection optics 256 (e.g., lens, mirrors) may focus the light pulse to a specific point on the sample, thereby creating a plasma plume. The focusing and light collection optics 256 may also capture/collect the light pulse/beam to avoid light interference. The collected light pulse is then passed through detector 258 (e.g., spectrometer) to obtain sample measurements (e.g., spectral data). For example, detector 258 may disperse the light pulse into individual wavelengths, determine the intensity of light at each wavelength (e.g., using a charge-coupled device (CCD) or photo-multiplier tube (PMT)), and generate a spectrum that is characteristic of specific elements in the sample.

Data acquisition & analysis component 260 may include computing hardware such as a central processing unit, memory, and storage, as described in the computer system of FIG. 7. Data acquisition & analysis component 260 is configured to receive the measurement data from detector 258 and perform the elemental composition analysis on the measurement data. In some embodiments, LIBS device 204 may also communicate with an alternative or additional data processing unit (e.g., 206) to perform LIBS analysis and any other elemental identification and quantification analyses. Based on the analysis, a result is generated in 262. For example, the result may be a report or graphical output showing the elemental composition of the sample. Various types of measurements and analyses may be conducted by the data analysis subsystem including data acquisition & analysis component 260 and data processing unit 206, which will be further described below with reference to FIGS. 3-6.

The present system may deploy one or more improved LIBS devices to implement the disclosed functionalities. For example, the improved LIBS device (e.g., 204) may be equipped, attached, or associated with an autofocusing component to enable consistent, in-focus measurements of samples without moving other optical components. The autofocusing component may be included as a part of a suitable optical assembly that is hand-held, attached to a mobile vehicle, or integrated with a positioning system (e.g., robotic arm, boom, leg, conveyor belt), etc. Using the autofocusing component, spectroscopy measurements can be automatically focused based on a focal length of its optics relative to the target sample. Instead of using certain mechanisms (e.g., mechanical actuators) to change the position of the optical component(s), in some embodiments, the autofocusing component may include a shape changing lens (e.g., variable focus lens) to allow continuous tuning of a focal length and working distance of the autofocusing component relative to a target sample.

### Data Acquisition and Evaluation Using LIBS Device

As discussed above, components 252, 254, 256, 258, and/or 260 of LIBS device 204 may work together with other units of data system 200 to acquire sample data and provide insights about the elemental composition of a sample (e.g., soil sample). In the illustrated embodiment of FIG. 3, one or more LIBS devices 204 may be used to determine weathering status 304, evaluate specific soil contents/elements 306, calculate heavy metal concentration 308, evaluate metal ratios in soils 310, and/or measure soil organic carbon 312. In some embodiments, one or more LIBS devices 200 may also be used to measure and analyze the compositional characteristics of soils, the biological composition of plant and seed matter, and the elemental composition of rock samples.

In some embodiments, the present disclosure describes a LIBS device (e.g., 204) used for determining weathering status 304. In some embodiments, a LIBS-based device may be employed to assess the composition of lighter cations relative to heavier cations in applied rock dust. This composition analysis may be used in determining the weathering status of the rock dust application on agricultural fields, pasture lands, or any other natural environments. Thus, an approach is obtained, for determining the loss of rock dust due to enhanced rock weathering (ERW) for the purpose of monitoring, reporting, and verification (MRV) to create carbon credits.

In some embodiments, the present disclosure describes a LIBS device (e.g., 204) for evaluating specific soil elements 306, for example, the nitrogen (N), phosphorus (P), and potassium (K) content of the soil. This is the estimation of nutrient requirements, factoring in additional nutrients provided by rock dust application. The use of LIBS devices in elemental composition analysis can reduce farmers' burden to apply fertilizers (i.e., NPK nitrogen-phosphorus-potassium) while conducting ERW projects. LIBS allows the quantification of these additional bioavailable nutrients, which become active in the soil column with the breakdown of the rock minerals (e.g., being modified through chemical, physical, or biological interactions with rocks and minerals).

In some embodiments, the present disclosure describes a LIBS device (e.g., 204) for calculating heavy metal concentrations 308, e.g., the amounts of metallic elements (often toxic) found in the soils or rock dust used for ERW. These elements may include metals such as lead (Pb), mercury (Hg), nickel (ni), etc., and can be found in pesticides, fertilizers, or untreated sewage sludge introduced into the soil. The heavy metal may accumulate in the environment to pose serious health and ecological risks. The disclosed LIBS device may provide insights into the safety considerations of cultivating various crops, for example, providing insights into phytoremediation such as using sunflowers to remove Strontium, which is poisonous metal to most other livings. According to US Environmental Protection Agency (EPA) standards, the bio-availability of metals and dissolved aqueous metals will be needed for calculating heavy metal concentrations.

In some embodiments, the present disclosure describes a LIBS device (e.g., 204) for evaluating key metal ratios in soils for ERW 310. In some embodiments, critical metal ratios in soils are assessed using LIBS technology before the implementation of ERW (i.e., before the rock dust application for enhanced weathering). This process can be outlined as follows:
i. Sample Collection: soil samples are collected from the target area where ERW is planned. These samples represent the current soil composition in the region;
ii. LIBS Analysis: the collected soil samples are subjected to LIBS analysis. The LIBS device measures the elemental composition of the soil, providing baseline data on the concentrations of various metals being present;
iii. Trace metal ratios and potential pulverized rock dust field amendments may then be evaluated together to determine if the rock dust is appropriate for application to the field; and
   a. Evaluate environmental concerns,
   b. Evaluate the potential for signal strength in measurements: measuring key metal ratios for MRV process.
iv. Key Metal Ratios: specific metal ratios of interest, which are known to influence the effectiveness of ERW, are calculated based on the LIBS data. These key metal ratios often include ratios such as calcium to magnesium (Ca/Mg), calcium to potassium (Ca/K), or calcium to sodium (Ca/Na), among others.

In some embodiments, the calculated key metal ratios are evaluated to determine the suitability of the soil for ERW. An ideal balance of these ratios is typically necessary for optimal carbon capture and weathering outcomes. The assessment results may guide decisions regarding the need for rock dust application and the specific rock dust type required to achieve the desired metal ratios for effective ERW.

By employing LIBS technology for the evaluation of key metal ratios in soils before rock dust application for enhanced weathering, informed decision-making can be used to ensure that the chosen ERW strategy is tailored to the specific soil composition of the target area. This optimization may also contribute to efficient carbon dioxide removal and MRV.

In some embodiments, the present disclosure describes a LIBS device (e.g., 204) for measuring soil organic carbon 312. A LIBS device can be used to measure soil organic carbon (SOC) levels. The SOC level represents the amount of carbon stored in the form of organic compounds in the soil, and SOC is critical to soil quality and carbon sequestration because it helps store carbon from the atmosphere and contributes to mitigating climate change. By measuring the SOC level, the present disclosure facilitates the correlation between changes in organic carbon attributable to rock dust application, thereby offering valuable insights into soil improvement. In some embodiments, soil organic carbon measurements may be approximated by the loss of material on ignition (LOI) (e.g., by measuring the weight loss of organic matter content after heating the sample to a high temperature).

### LIBS Analysis

Numerous LIBS analyses may be conducted based on element measurements, calculation, and evaluation. In addition to in-depth soil analysis, when a LIBS device is used to measure the biological composition of plant and seed matter, the LIBS analysis can serve to validate the absence of potential metal accumulation resulting from enhanced weathering processes.

In some embodiments, a LIBS device may also be used for rock sample analysis, where the elemental composition of raw rock samples from rock quarries or in the field is analyzed. This LIBS analysis may be applied to determine an enhanced weathering potential and a total carbon dioxide removal (CDR) potential. The enhanced weathering potential is the amount of atmospheric carbon dioxide that can be removed and stored through the ERW process (e.g., manual application of rocks or minerals) over a specific period of time. This factor may vary depending on the type of rocks/minerals used in ERW, the surface area available for reaction, environmental conditions (e.g., precipitation, temperature), etc. The total CDR potential is the amount of carbon dioxide that can be removed from the atmosphere in the long term, which depends on the effectiveness of the ERW process.

In some embodiments, a spectral fluorescence device may be used in combination with LIBS analysis to create a dual system. This dual system enables the simultaneous determination of organic carbon and elemental composition in samples. This technology is particularly useful for assessing the composition of lighter cations relative to heavier cations, aiding in the determination of rock dust weathering status. In some embodiments, this approach may also be used to help refine soil organic carbon estimates outlined in 312 of FIG. 3. In general, the combination of LIBS and fluorescence may expand and strengthen the LIBS measurement and analysis, e.g., as discussed in FIG. 3.

### Beneficial Features of LIBS Device and Analysis

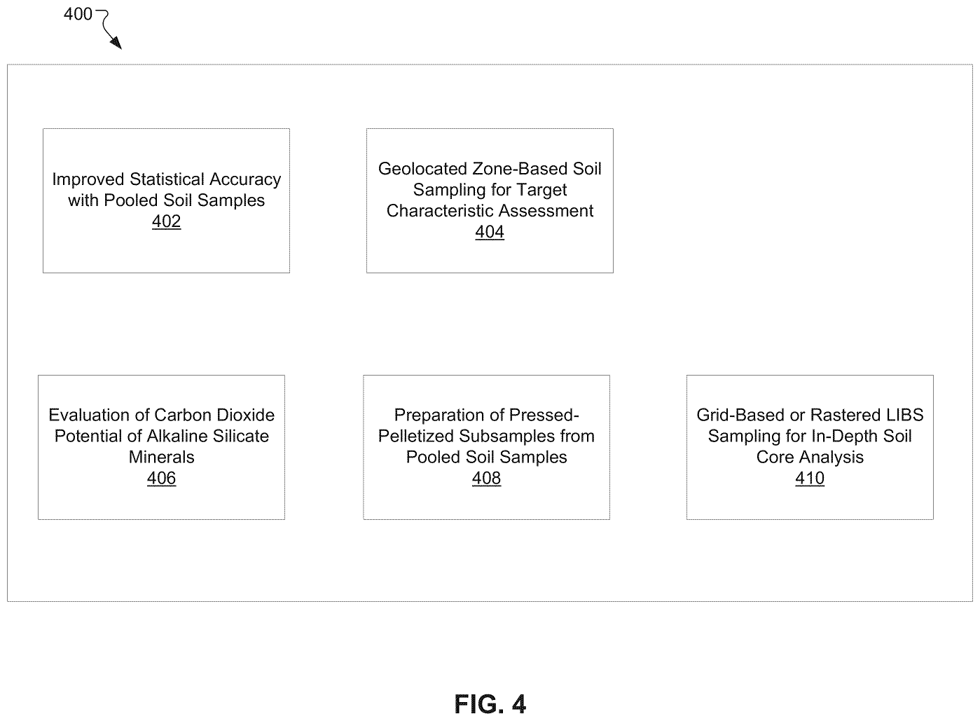

FIG. 4 illustrates a block diagram 400 of exemplary features of LIBS devices and analysis used in the MRV process for ERW as described in the present system.

The present system allows improved statistical accuracy with pooled/composite soil samples, as shown in 402. By aggregating soil samples from various locations within a field, the present system seeks to accurately determine the central compositional tendency and associated dispersion or variance in the soil's elemental composition. As compared to the current techniques, the utilization of pooled samples enables a more robust and representative analysis of the soil's overall composition, contributing to a more precise assessment of soil quality and environmental conditions. The accurate measurement of pooled samples is particularly important to soil fertility assessment, soil quality monitoring, and precision agriculture.

The present system provides geolocated zone-based soil sampling for target characteristic assessment, as shown in 404. In some embodiments, the present system allows the collection of soil samples from specific geolocated zones within a field. By strategically pooling samples from these distinct zones, the analysis can yield precise data regarding the central tendency and variance of the target characteristic within each zone. This geolocated zone-based sampling approach offers a granular understanding of the spatial heterogeneity, distribution, and variability of the target characteristic, thereby enabling informed decision-making for soil management and ERW applications. Potential future obstacles with a MRV process based on soil heterogeneity may also be addressed by examining measurements at this juncture. In some embodiments, zones may be determined based on soil type, gridded areas, etc. A zone may also be small, predefined regions that include, but are not limited to, a circular region with a predefined radius or a square area with a predefined side length.

The LIBS device of the present system can be used in rock sample analysis to determine enhanced weathering potential and total carbon dioxide removal (CDR) potential. In particular, the present system may evaluate the carbon dioxide potential of alkaline silicate minerals, as shown in 406. The present system can assess the carbon dioxide potential of alkaline silicate minerals including, but not limited to, basalt, olivine, and wollastonite. These minerals are known for their capacity to sequester carbon dioxide through enhanced weathering processes. In some embodiments, the present system may utilize a LIBS device to analyze the elemental composition of these minerals and determine their specific potential for CDR. By accurately quantifying the CDR potential of alkaline silicate minerals, the present system gains valuable insights into the efficacy of a CDR solution.

The present system allows systematic preparation of pressed-pelletized subsamples derived from pooled soil samples, as shown in 408. By compressing and pelletizing soil material from each pooled sample, the present system may create a consistent and standardized substrate for LIBS analysis. This process can minimize variations in sample presentation, ensuring reliable and reproducible results during LIBS analysis. This process further streamlines the handling of samples, allowing for an efficient assessment of the central compositional tendency and variance of the target characteristics within the soil, and ultimately enhancing the accuracy of soil quality and environmental assessments.

The present system further allows grid-based or rastered LIBS sampling for in-depth soil core analysis, as shown in 410. In some embodiments, the present system may conduct grid-based or rastered analyses of the pelletized subsamples, where the LIBS device systematically scans and analyzes discrete regions or points on the pellet surface in a structured grid or raster pattern. This approach enables a comprehensive examination of the intersample heterogeneity (or microheterogeneity) of soil characteristics and the distribution of elemental composition within the subsamples.

Through this analysis, the present system may achieve a high-resolution understanding of the central compositional tendency and variance of the target characteristics in the soil, and the detailed information may provide invaluable insights into the spatial variability of soil properties, thereby enhancing precision in soil quality assessment and MRV for CDR applications.

Using rasterized or gridded LIBS sampling can also systematically measure the elemental composition of intact soil cores at various depths. By taking measurements in a prescribed structured grid or raster pattern for LIBS analysis along the length of an intact soil core, the present approach enables precise characterization of the compositional variations with depth. This is particularly beneficial for understanding how soil properties change at different depths within the core, offering crucial insights into subsurface composition and environmental conditions. The present system seeks to assess soil heterogeneity and geological attributes with depth, contributing to informed decisions in ERW applications and MRV techniques.

### Device Characteristics and Deployment

The present system supports specific device characteristics and deployment that are beneficial to the MRV process for ERW. In some embodiments, the present system supports handheld and miniaturized LIBS devices for soil sampling, field-based depth-integrated soil profiling with LIBS and soil auger, and in-field deployment of LIBS technology on motor vehicles, etc.

The LIBS device described herein can be a handheld device that is portable, easy to operate, and suitable for rapid data acquisition. This handheld device is particularly useful in patchwork small-holder farms (e.g., rice paddies). It is engineered to provide a simplified user interface while maintaining the analytical accuracy and precision required for soil and environmental analysis. Any instantiation of the LIBS device may contain fewer optical sensors than the original, making the device more affordable and lighter. As a result, this LIBS device is adaptive for use in rural, remote, and economically disadvantaged communities. In addition, the miniaturized form of the device enables the present system to perform real-time, in-situ analyses, contributing to timelier and data-driven decision-making processes in agriculture, geology, and environmental science.

The present system also allows a LIBS device to be combined with a soil auger to obtain a depth-integrated soil profile of a predetermined target characteristic. A soil auger is employed to collect soil samples at various depths within the field. These samples are representative of the soil composition at specific depth intervals.

After and/or during the auguring process, the present system performs LIBS analysis on the collected soil samples. A LIBS device can rapidly and accurately measure the elemental composition of the soil samples, including the concentration of the target characteristic of interest. The present system may then integrate the LIBS data obtained from each depth interval to construct a depth-integrated soil profile of the target characteristic. This profile provides a comprehensive understanding of how the target characteristic varies with depth within the soil.

A LIBS device/instrument can be deployed in different ways. In some embodiments, the present system supports in-field deployment of LIBS technology on motor vehicles. LIBS is integrated into motor vehicles, such as tractors or other agricultural machinery, for in-field deployment. The present system seamlessly integrates LIBS instrumentation into the design of motor vehicles used for field operations, such as tractors.

The LIBS device can be securely mounted, ensuring stability during vehicle movement. The LIBS device may operate in real time as the motor vehicle traverses the field. The LIBS device continuously analyzes the soil or other target materials as the vehicle moves, providing instant elemental composition data. The present system may collect LIBS-generated data and integrate the data into a data acquisition (DAQ) onboard the vehicle, allowing for real-time decision-making based on soil or material composition. A vehicle operator can use the LIBS data to make immediate adjustments to field operations for any ERW-related purpose, such as varying fertilizer application rates or adjusting rock dust application rates based on the in-field analysis. The present system may log the LIBS data, providing a detailed record of the soil or material composition across the field for subsequent analysis and optimization.

In some embodiments, the present system may support other deployment methods. For example, one or more LIBS devices may be integrated into drones, underwater vehicles/amphibious, or other mobil instruments.

### Data Analysis and Modeling

FIG. 5 illustrates a block diagram 500 of exemplary features of data analysis and modeling used in the present system. As depicted in FIG. 5, the data analysis and modeling 502 of the present system may be featured with various analytical tools such as multivariate distribution modeling for cation covariance analysis 504, empirical modeling for weathering prediction and carbon credit creation 506, calibration with high-precision inductively coupled plasma mass spectrometers (ICP-MS) data 508, construction of calibration curves for soil-rock dust mixtures with high-precision ICP-MS 510, and collaborative spot-checking and uncertainty modeling with ICP-MS and LIBS for CDR MRV 512. In some embodiments, these data analyses and modeling may be performed at least by data processing unit 206 in combination with data acquisition & analysis component 260 as shown in FIGS. 2A and 2B (i.e., data analysis subsystem).

The present system provides multivariate distribution modeling for cation covariance analysis, as shown in 504. In some embodiments, the LIBS data can be leveraged for analyzing and modeling the covariance relationships among key cations in soil including, but not limited to, titanium, calcium, sodium, magnesium, potassium, phosphorous, and nitrogen. In some embodiments, the present system entails fitting multivariate normal or non-normal distributions to the data obtained through LIBS measurements.

Multivariate distribution fitting allows for the comprehensive assessment of the covariance and correlations between key cations in soil. It provides valuable insights into the simultaneous variations and relationships among these elements, contributing to a deep understanding of soil composition and baseline information required for MRV. The present system uses LIBS to provide dense and rapid enough data to find systematic variances in soil heterogeneity within any given field, pasture, agricultural setting, or any other natural setting.

In some embodiments, the present system may apply the quantitative elemental analysis approach as described in Sobron, titled “non-linear methods for quantitative elemental analysis and mineral classification using laser-induced breakdown spectroscopy (LIBS),” U.S. patent application Ser. No. 15/998,391, which is hereby incorporated by reference in its entirety.

In some embodiments, the present system supports empirical modeling for weathering prediction and carbon credit creation, as shown in 406. That is, data collected in the present system can be leveraged to develop an empirically driven model for predicting the weathering of rock dust applied to fields and providing suggestions and instructions for subsequent ERW implementation.

The empirical model may help the monitoring, reporting, and verification (MRV) process, which aims at quantifying the carbon capture potential resulting from enhanced weathering practices. By using dense, LIBS-generated data to create a predictive model, it becomes possible to monitor and report on the efficiency of carbon capture using regression-based and/or machine-learning methods. As a result, the time between data collection and sales for creating carbon credits may be reduced. The empirically driven model therefore facilitates accurate and data-driven MRV processes while incentivizing an easy switch to practices where carbon credits may be created through ERW. For example, when structure a predictive model, the present system may use mineral type, application rate, surface area of mineral, climate data, and environmental carbon dioxide concentration as input parameters of the model. The present system may calculate the weathering rate based on the environmental conditions and mineral properties, calculate the sequestration of carbon dioxide, and use weathering rate equations to predict how much carbon dioxide may be captured within a specific area over a specific timeframe. The present system may also calculate the total carbon dioxide removal rate to estimate global or regional carbon dioxide sequestration potential.

To ensure accuracy and precision, in some embodiments, the present system may calibrate the LIBS device using high-precision measurements obtained from ICP-MS, as shown in 508.

ICP-MS represents a well-established and highly accurate analytical technique, known for its exceptional precision in measuring elemental composition. By calibrating the LIBS device against ICP-MS data, the present system may establish a robust and reliable calibration framework. This calibration method may serve to validate and fine-tune the LIBS measurements, ensuring accuracy and traceability to a gold standard in elemental analysis.

The integration of ICP-MS calibration in the present system enhances the trustworthiness and consistency of LIBS measurements, and the enhanced measurements may be suitable for a wide range of ERW applications for MRV. This calibration approach boosts the credibility of LIBS technology in scientific research and regulatory compliance (e.g., metals contamination according to EPA standards).

The present system supports the construction of calibration curves for soil-rock dust mixtures with high-precision ICP-MS, as shown in 510. In some embodiments, the present system may use known mixtures of soil and rock dust to create calibration curves. With a soil sample and rock dust sample that have already been characterized via ICP-MS or other analytical techniques, the present system can obtain known mixtures such that the rock dust comprises a known percentage of the weight of a newly created sample. By testing many mixed samples using LIBS with an array of rock dust ranging from 0 to 100% weight by mass, the present system can determine a series of calibration curves. These curves may fit the compositional characteristics of a particular soil type-rock dust combination. The present system may then store the data for soil type-rock dust combinations, creating a large database of possible LIBS spectral signals for a range of possible compositional arrays. With a sufficiently large database, the present system can apply one or more regression-based, machine-learning-based tools, and principal component analysis to select the most optimal calibration curve for new measurement(s).

Here, the present system aims to strengthen the credibility of carbon credit generation. In some embodiments, the present system may use the misfit of measurements to known calibration curves to quantify measurement uncertainty, so that carbon credit sales effectively incorporate potential variations in carbon capture into pricing structures.

In some embodiments, the present system supports collaborative spot-checking and uncertainty modeling with ICP-MS and LIBS for CDR MRV, as shown in 512. In other words, ICP-MS and LIBS are used collaboratively to spot-check and validate the empirically driven model developed for CDR MRV. This process ensures that the model accurately reflects the carbon capture and sequestration processes in real-world applications, enhancing the reliability of carbon credit generation. The present system may also use the same collaborative framework to generate uncertainty models when upscaling the sample area for MRV. To upscale the sample area for MRV, test plots are used to make projections for larger fields, and/or several fields are used to project CDR for a region, etc. By systematically assessing variations and uncertainties using ICP-MS and LIBS data, these models provide insights into the potential variability in carbon capture estimates as the sample area is expanded. This information is essential for accurately quantifying carbon credits and assuaging buyers' concerns about the quality of carbon credits. The information is also crucial to assess the quality of MRV.

The present system aims to strengthen the credibility of carbon credit generation and improve informed decision-making in ERW-related land management. By integrating uncertainty modeling, the present system enhances its ability to account for variations in carbon capture and carbon dioxide removal potential and provides a comprehensive assessment of carbon dioxide removal efforts. The present system may enhance the credibility of carbon credit generation while providing transparency and accountability in environmental initiatives.

### Flowchart

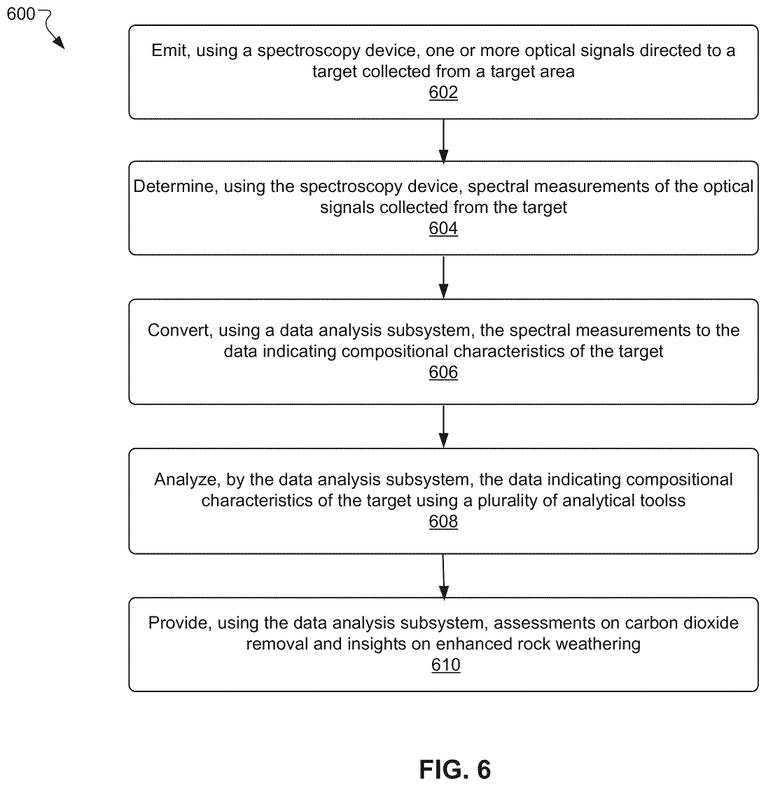

FIG. 6 illustrates a flowchart 600 of monitoring, reporting, and verification of enhanced rock weathering, according to some embodiments. Method 600 may be implemented using a LIBS-based data system 200, as shown in FIGS. 2A and 2B. A spectroscopy device (e.g., LIBS device 204) is used to obtain measurements from a target sample. At step 602, the spectroscopy device (e.g., using optical source 254) emits one or more optical signals directed to a target collected from a target area (e.g., managed field or other agricultural settings). At step 604, the spectroscopy device (e.g., using detector 258) determines spectral measurements of the optical signals collected from the target. The target can be a pooled sample that includes soil samples collected and combined from multiple locations within the target area. One or more geolocated zones within the target area may be identified (e.g., by sample handing unit 202) from which the pooled sample is collected, and the pooled sample is collected from the target area using grid-based or rastered LIBS sampling.

As shown in FIGS. 2A and 2B, a data analysis subsystem (e.g., including data processing unit 206 and data acquisition & analysis component 260) is communicatively coupled to the spectroscopy device (e.g., LIBS device 204). At step 606, the data analysis subsystem is configured to convert the spectral measurements to data indicating compositional characteristics of the target, which includes at least one or more of determining a weathering status, evaluating target contents, calculating heavy metal concentration, determining metal ratios in the target, or measuring organic carbon in the target, are performed. For example, to determine the weathering status, loss of rock dust may be determined by assessing the composition of cations in rock dust applied to the target area through the enhanced rock weathering.

The data analysis subsystem is then configured to analyze the data indicating compositional characteristics of the target using a plurality of analytical tools at step 608, and provide assessment on carbon dioxide removal and insights on enhanced rock weathering at step 610. In some embodiments, to analyze the compositional data, the data analysis subsystem uses various analytical tools to perform one or more of multivariate distribution modeling for cation covariance analysis, empirical modeling for weathering prediction and carbon credit creation, calibration with high-precision ICP-MS data, construction of calibration curves for soil-rock dust mixtures with high-precision ICP-MS, and collaborative spot-checking and uncertainty modeling with ICP-MS and LIBS, etc. To provide the assessment, the data analysis subsystem may also calculate one or more of an enhanced weathering potential and a total carbon dioxide removal (CDR) potential.

### Computer Implementation

In some examples, some or all of the processing described above can be carried out on a personal computing device, on one or more centralized computing devices, or via cloud-based processing by one or more servers. Some types of processing can occur on one device and other types of processing can occur on another device. Some or all of the data described above can be stored on a personal computing device, in data storage hosted on one or more centralized computing devices, and/or via cloud-based storage. Some data can be stored in one location and other data can be stored in another location. In some examples, quantum computing can be used, and/or functional programming languages can be used. Electrical memory, such as flash-based memory, can be used.

FIG. 7 is a block diagram of an example computer system 700 that may be used in implementing the technology described herein. General-purpose computers, network appliances, mobile devices, or other electronic systems may also include at least portions of the system 700. The system 700 includes a processor 710, a memory 720, a storage device 730, and an input/output device 740. Each of the components 710, 720, 730, and 740 may be interconnected, for example, using a system bus 750. The processor 710 is capable of processing instructions for execution within the system 700. In some implementations, the processor 710 is single-threaded. In some implementations, the processor 710 is a multi-threaded processor. The processor 710 is capable of processing instructions stored in the memory 720 or on the storage device 730.

Memory 720 stores information within the system 700. In some implementations, the memory 720 is a non-transitory computer-readable medium. In some implementations, the memory 720 is a volatile memory unit. In some implementations, the memory 720 is a non-volatile memory unit.

The storage device 730 is capable of providing mass storage for the system 700. In some implementations, the storage device 730 is a non-transitory computer-readable medium. In various implementations, the storage device 730 may include, for example, a hard disk device, an optical disk device, a solid-state drive, a flash drive, or some other large-capacity storage device. For example, the storage device may store long-term data (e.g., database data, file system data, etc.). The input/output device 740 provides input/output operations for the system 700. In some implementations, the input/output device 740 may include one or more network interface devices, e.g., an Ethernet card, a serial communication device, e.g., an RS-232 port, and/or a wireless interface device, e.g., an 802.11 card, a 3G wireless modem, or a 4G wireless modem. In some implementations, the input/output device may include driver devices configured to receive input data and send output data to other input/output devices, e.g., keyboard, printer, and display devices 760. In some examples, mobile computing devices, mobile communication devices, and other devices may be used.

In some implementations, at least a portion of the approaches described above may be realized by instructions that upon execution cause one or more processing devices to carry out the processes and functions described above. Such instructions may include, for example, interpreted instructions such as script instructions, executable code, or other instructions stored in a non-transitory computer-readable medium. The storage device 730 may be implemented in a distributed way over a network, such as a server farm or a set of widely distributed servers, or may be implemented in a single computing device.

Although an example processing system has been described in FIG. 7, embodiments of the subject matter, functional operations, and processes described in this specification can be implemented in other types of digital electronic circuitry, in tangibly embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible nonvolatile program carrier for execution by, or to control the operation of, data processing apparatus. Alternatively or in addition, the program instructions can be encoded on an artificially generated propagated signal, e.g., a machine-generated electrical, optical, or electromagnetic signal that is generated to encode information for transmission to a suitable receiver apparatus for execution by a data processing apparatus. The computer storage medium can be a machine-readable storage device, a machine-readable storage substrate, a random or serial access memory device, or a combination of one or more of them.

The term “system” may encompass all kinds of apparatus, devices, and machines for processing data, including by way of example a programmable processor, a computer, or multiple processors or computers. A processing system may include special-purpose logic circuitry, e.g., an FPGA (field programmable gate array) or an ASIC (application-specific integrated circuit). A processing system may include, in addition to hardware, code that creates an execution environment for the computer program in question, e.g., code that constitutes processor firmware, a protocol stack, a database management system, an operating system, or a combination of one or more of them.

A computer program (which may also be referred to or described as a program, software, a software application, a module, a software module, a script, or code) can be written in any form of programming language, including compiled or interpreted languages, or declarative or procedural languages, and it can be deployed in any form, including as a standalone program or as a module, component, subroutine, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable computers executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application-specific integrated circuit).

Computers suitable for the execution of a computer program can include, by way of example, general or special purpose microprocessors or both, or any other kind of central processing unit. Generally, a central processing unit will receive instructions and data from a read-only memory, a random access memory, or both. A computer generally includes a central processing unit for performing or executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few.

Computer-readable media suitable for storing computer program instructions and data include all forms of non-volatile memory, media, and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in special-purpose logic circuitry.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's user device in response to requests received from the web browser.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), e.g., the Internet.

The computing system can include clients and servers. A client and server are generally remote from each other and typically interact through a communication network. The relationship between client and server arises by virtue of computer programs running on the respective computers and having a client-server relationship with each other.

While this specification contains many specific implementation details, these should not be construed as limitations on the scope of what may be claimed, but rather as descriptions of features that may be specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous. Other steps or stages may be provided, or steps or stages may be eliminated, from the described processes. Accordingly, other implementations are within the scope of the following claims.

### Terminology

The phraseology and terminology used herein are for the purpose of description and should not be regarded as limiting.

The term “approximately”, the phrase “approximately equal to”, and other similar phrases, as used in the specification and the claims (e.g., “X has a value of approximately Y” or “X is approximately equal to Y”), should be understood to mean that one value (X) is within a predetermined range of another value (Y). The predetermined range may be plus or minus 20%, 10%, 5%, 3%, 1%, 0.1%, or less than 0.1%, unless otherwise indicated.

The indefinite articles “a” and “an,” as used in the specification and in the claims, unless clearly indicated to the contrary, should be understood to mean “at least one.” The phrase “and/or,” as used in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The use of “including,” “comprising,” “having,” “containing,” “involving,” and variations thereof, is meant to encompass the items listed thereafter and additional items.

Use of ordinal terms such as “first,” “second,” “third,” etc., in the claims to modify a claim element does not by itself connote any priority, precedence, or order of one claim element over another or the temporal order in which acts of a method are performed. Ordinal terms are used merely as labels to distinguish one claim element having a certain name from another element having a same name (but for use of the ordinal term), to distinguish the claim elements.

Each numerical value presented herein, for example, in a table, a chart, or a graph, is contemplated to represent a minimum value or a maximum value in a range for a corresponding parameter. Accordingly, when added to the claims, the numerical value provides express support for claiming the range, which may lie above or below the numerical value, in accordance with the teachings herein. Absent inclusion in the claims, each numerical value presented herein is not to be considered limiting in any regard.

The terms and expressions employed herein are used as terms and expressions of description and not of limitation, and there is no intention, in the use of such terms and expressions, of excluding any equivalents of the features shown and described or portions thereof. In addition, having described certain embodiments of the invention, it will be apparent to those of ordinary skill in the art that other embodiments incorporating the concepts disclosed herein may be used without departing from the spirit and scope of the invention. The features and functions of the various embodiments may be arranged in various combinations and permutations, and all are considered to be within the scope of the disclosed invention. Accordingly, the described embodiments are to be considered in all respects as only illustrative and not restrictive. Furthermore, the configurations, materials, and dimensions described herein are intended as illustrative and in no way limiting. Similarly, although physical explanations have been provided for explanatory purposes, there is no intent to be bound by any particular theory or mechanism, or to limit the claims in accordance therewith.

What is claimed is:

1. A system for monitoring, reporting, and verification of enhanced rock weathering comprising:
a spectroscopy device comprising an optical source and a detector, wherein the optical source is configured to emit one or more optical signals directed to a target collected from a target area, and the detector is configured to determine spectral measurements of the optical signals collected from the target; and
a data analysis subsystem coupled to the spectroscopy device and configured to:
convert the spectral measurements to data indicating compositional characteristics of the target;
analyze the data indicating compositional characteristics of the target using a plurality of analytical tools; and
provide assessments on carbon dioxide removal and insights on enhanced rock weathering.

2. The system of claim 1, wherein, to convert the spectral measurements to the data indicating compositional characteristics of the target, the data analysis subsystem is further configured to determine a weathering status, evaluate target contents, calculate heavy metal concentration, determine metal ratios in the target, or measure organic carbon in the target.

3. The system of claim 2, wherein, to determine the weathering status, the data analysis subsystem is further configured to determine loss of rock dust by assessing composition of cations in rock dust applied to the target area through the enhanced rock weathering.

4. The system of claim 2, wherein the heavy metal concentration is calculated in response to receiving bio-availability of metals and dissolved aqueous metals.

5. The system of claim 2, wherein the soil organic carbon is measured based on determining loss of material on ignition.

6. The system of claim 1, wherein the target is a pooled sample that includes soil samples collected and combined from multiple locations within the target area.

7. The system of claim 6, further comprising a sampling handing unit configured to identify geolocated zones within the target area from which the pooled sample is collected, wherein the pooled sample is collected from the target area using grid-based or rastered laser induced breakdown spectroscopy (LIBS) sampling.

8. The system of claim 1, wherein, to analyze the data indicating compositional characteristics, the data analysis subsystem is further configured to perform one or more of multivariate distribution modeling for cation covariance analysis, empirical modeling for weathering prediction and carbon credit creation, calibration with high-precision inductively coupled plasma mass spectrometers (ICP-MS) data, construction of calibration curves for soil-rock dust mixtures with high-precision ICP-MS, and collaborative spot-checking and uncertainty modeling with ICP-MS and LIBS.

9. The system of claim 1, wherein, to provide the assessments on carbon dioxide removal and insights on enhanced rock weathering, the data analysis subsystem is further configured to create a prediction model to provide data support for subsequent implementation of enhanced rock weathering.

10. The system of claim 1, wherein, to provide the assessments on carbon dioxide removal and insights on enhanced rock weathering, the data analysis subsystem is further configured to determine one or more of an enhanced weathering potential and a total carbon dioxide removal (CDR) potential.

11. The system of claim 1, wherein the spectroscopy device is a handheld LIBS device.

12. The system of claim 1, wherein the spectroscopy device is a LIBS device that is mounted on a motor vehicle and operates in real time as the motor vehicle traverses the target area, and wherein the data analysis subsystem is integrated onboard the motor vehicle for real-time data acquisition and analysis.

13. The system of claim 1, wherein the spectroscopy device is a LIBS device that is combined with a soil auger to obtain a depth-integrated soil profile of a predetermined target characteristic from the target.

14. A method for monitoring, reporting, and verification of enhanced rock weathering comprising:
emitting, using a spectroscopy device, one or more optical signals directed to a target collected from a target area;
determining, using the spectroscopy device, spectral measurements of the optical signals collected from the target; and
converting, using a data analysis subsystem, the spectral measurements to data indicating compositional characteristics of the target;
analyzing, by the data analysis subsystem, the data indicating compositional characteristics of the target using a plurality of analytical tools; and
providing, using the data analysis subsystem, assessments on carbon dioxide removal and insights on enhanced rock weathering.

15. The method of claim 14, wherein converting the spectral measurements comprises one or more of determining a weathering status, evaluating target contents, calculating heavy metal concentration, determining metal ratios in the target, or measuring organic carbon in the target.

16. The method of claim 15, wherein determining the weathering status comprises determining loss of rock dust by assessing composition of cations in rock dust applied to the target area through the enhanced rock weathering.

17. The method of claim 14, further comprising identifying geolocated zones within the target area from which the target is collected, wherein the target is collected from the target area using grid-based or rastered laser induced breakdown spectroscopy (LIBS) sampling.

18. The method of claim 14, wherein analyzing the data indicating compositional characteristics comprises performing one or more of multivariate distribution modeling for cation covariance analysis, empirical modeling for weathering prediction and carbon credit creation, calibration with high-precision inductively coupled plasma mass spectrometers (ICP-MS) data, construction of calibration curves for soil-rock dust mixtures with high-precision ICP-MS, and collaborative spot-checking and uncertainty modeling with ICP-MS and LIBS.

19. The method of claim 14, wherein providing the assessments on carbon dioxide removal and insights on enhanced rock weathering comprises determining one or more of an enhanced weathering potential and a total carbon dioxide removal (CDR) potential.

20. The method of claim 14, wherein the spectroscopy device is a LIBS device that is mounted on a motor vehicle and operates in real time as the motor vehicle traverses the target area, and wherein the data analysis subsystem is integrated onboard the motor vehicle for real-time data acquisition and analysis.
